(19) United States
(12) Patent Application Publication
(10) Pub. No.: US 2012/0003178 A1
(43) Pub. Date: Jan. 5, 2012

(54) EVALUATING THE THERAPEUTIC POTENTIAL OF A GLUCAN

(75) Inventor: Reinhard Koenic, Rockville, MD (US)

(73) Assignee: NOVOGEN RESEARCH PTY LTD, WALES (AU)

(21) Appl. No.: 12/918,201

(22) PCT Filed: Feb. 19, 2009
(86) PCT No.: PCT/AU09/00186

§ 371 Date: Nov. 16, 2010

Related U.S. Application Data
(60) Provisional application No. 61/029,758, filed on Feb. 19, 2008.

(51) Int. Cl. C12Q 1/68; A61M 37/00; A61K 38/19; A61K 38/18; C12N 5/071; G01N 33/53

(52) U.S. Cl. 424/85.1; 435/6.11; 435/6.13; 435/375; 435/7.92; 514/7.6; 604/522

(57) ABSTRACT

Provided herein are methods for predicting the response of a subject to administration of a glucan prior to said administration, comprising in one embodiment: isolating at least one cell from the subject, wherein the cell is capable of being stimulated by the glucan; contacting the at least one cell with the glucan and incubating for a period of time and under suitable conditions sufficient to stimulate the cell(s) thereby inducing a change in the level of expression, activity and/or secretion of one or more biomarkers from the cell(s); and determining the level of expression and/or secretion of the at least biomarker, wherein the level of expression, activity and/or secretion is predictive of the response of the subject to administration of the glucan.

### FIELD OF THE INVENTION

The present invention relates generally to methods for predicting the response of a subject to the administration of glucan. Thus, the invention also relates to the evaluation of the therapeutic potential and/or biological activity of glucans. Methods of the invention comprise the ex vivo stimulation of cells, such as macrophages, to alter the level of expression and/or secretion of various biomarker molecules. Methods of the invention are applicable to, inter alia, the generation of stimulated cells for administration to subjects in need of treatment.

### BACKGROUND OF THE INVENTION

Glucans are oligosaccharides or polysaccharides composed predominantly or wholly of glucose. Glucans are widely distributed in nature, being found in the cell walls of a variety of plants, fungi and microorganisms. Beta-(1,3)(1,6) glucans derived from yeast, such as the bakers yeast Saccharomyces cerevisiae, have been identified as having particular therapeutic potential for the treatment of a variety of disorders and conditions. Beta-glucans act to enhance the immune system, stimulating the activity of the primary defence cells, natural killer cells, neutrophils and macrophages. As such beta-glucans play a role in combating infection. Various beta-glucans have also been implicated in, for example, the treatment of cancer, septic shock, arthritis and in wound healing and reducing cholesterol.

A microparticulate beta-(1,3)(1,6) glucan from Saccharomyces cerevisiae, the isolation of which is described in U.S. Pat. No. 6,242,594, has been found to be therapeutically effective when administered, for example, to subjects suffering from a bone fracture, ulcers caused by physical trauma, surgical wounds, impaired blood flow, infections or neoplasia, or in persons in need of enhancement of fixation of implanted orthopaedic devices to bone. The glucan is also implicated in cosmetic skin surgery, tissue regeneration and tissue augmentation.

Accordingly, there is considerable interest in the development of pharmaceutical compositions comprising glucans.

A common problem facing the development of any composition designed for in vivo administration to a subject, in particular administration to humans, is the evaluation of the biological efficacy and therapeutic potential of the active ingredient(s). Biological efficacy and therapeutic potential can be adversely impacted by a number of factors. For example, many drugs and compositions are associated with side effects when administered to a subject. It is often difficult to ascertain the nature of such side effects in advance. Similarly, the therapeutic potential to be derived from any drug or composition can generally not be predicted with any confidence prior to administration. Quality assurance in the manufacturing process used to produce drugs and compositions for use on a clinical scale is of critical importance. There may be variability in activity between batches of any particular product and it is beneficial to have a reliable and simple means of detecting such variability.

Further to the above, it is becoming increasingly clear that the responses of different subjects to a particular drug or composition may differ, hence there is an increasing interest in so-called personalized medicine where treatments are tailored more specifically to the individual to be treated.

In view of such difficulties in the development of suitable therapeutics, there is a clear need for the development of simple and reliable methods for evaluating the biological activity and therapeutic potential of a drug or composition prior to its clinical application. For ethical reasons, it is apparent that such evaluations cannot be readily carried out on humans. There is also a significant trend against the use of animal models for such trials.

Currently used screening technologies for biological validation, pharmacological testing, and screening for success or failure of drugs and compositions in clinical trials typically suffer from a number of disadvantages including poor predictive value and absence of patient-specific focus.

Accordingly, there remains a clear need for the development of improved methods for the evaluation of biological activity and therapeutic potential. The present invention as disclosed herein provides suitable methods for evaluating the biological activity and therapeutic potential of glucans.

### SUMMARY OF THE INVENTION

According to a first aspect, the present invention provides a method for predicting the response of a subject to administration of a glucan prior to said administration, the method comprising:
(a) isolating at least one cell from the subject, wherein the cell is capable of being stimulated by the glucan;
(b) contacting the at least one cell with the glucan and incubating for a period of time and under suitable conditions sufficient to stimulate the cell(s) thereby inducing a change in the level of expression, activity and/or secretion of one or more biomarkers from the cell(s); and
(c) determining the level of expression and/or secretion of the at least biomarker,
   wherein the level of expression, activity and/or secretion is predictive of the response of the subject to administration of the glucan.

In a particular embodiment, the at least one cell capable of being stimulated by the glucan is a macrophage or precursor thereof. The precursor may be a monocyte. Accordingly, the at least one cell isolated from the subject may comprise monocytes that are subsequently differentiated in vitro prior to or during incubation with the glucan.

Typically the subject is a human. The subject may be suffering from any condition the treatment of which can be effected by administration of the glucan. By way of example only, the condition may be a skin wound or lesion, or a connective tissue disease or injury.

The stimulation of the cell(s) by the glucan may induce an increase or decrease in the level of expression, activity and/or secretion of the one or more biomarkers relative to the level detected in the absence of the glucan (unstimulated cell(s)). Thus, the method may comprise the further step of comparing the level of expression, activity and/or secretion of the one or more biomarkers in the presence of the glucan with the corresponding level of the same biomarker(s) in the absence of glucan, whereby the difference in the levels of expression, activity and/or secretion is predictive of the subject's response to administration of the glucan. The level of expression, activity and/or secretion in the absence of the glucan may be a predetermined control level.

Typically, the predicted response of the subject to administration of the glucan correlates with the biological activity of the glucan as determined by the level of expression, activity and/or secretion of the one or more biomarkers. Thus, the degree of change in the level of expression, activity and/or secretion of the biomarker(s) in the presence of the glucan is indicative of the biological activity and/or therapeutical potential of the glucan and predictive of the response of a subject to administration of the glucan.

Typically, the stimulation of the cells results in an increase in the level of expression, activity and/or secretion of the one or more biomarkers relative to the level of expression and/or secretion in the absence of the glucan.

The biomarker may be, for example, a cytokine, chemokine or growth factor. In one embodiment the biomarker is TNF-α. In a particular embodiment the cells are macrophages and the determining step (c) comprises determining the level of secretion of TNF-α from the macrophages.

The cells may be incubated in any suitable nutritive culture medium capable of sustaining the cells. The medium may include additional co-factors required or beneficial for expression, activity and/or secretion of the biomarker(s).

The cell(s) may be incubated with the glucan for between about 24 to 48 hours, more typically for about 24 hours.

The glucan may be derived from any suitable cellular source, such as yeast cell walls. The glucan may be a particulate or microparticulate glucan, such as a microparticulate branched beta-(1,3)(1,6) glucan. The glucan may be microparticulate poly-(1,3)-beta-D-glucopyranosyl-(1,6)-beta-D-glucopyranose.

The cell(s) may be re-introduced into the subject from whom they were isolated following the incubation step (b).

According to a second aspect, the present invention provides a method for evaluating the biological activity and/or therapeutic potential of a glucan, the method comprising:
(a) isolating at least one cell from a subject, wherein the cell is capable of being stimulated by the glucan;
(b) contacting the at least one cell with the glucan and incubating for a period of time and under suitable conditions sufficient to stimulate the cell(s) thereby inducing a change in the level of expression, activity and/or secretion of one or more biomarkers from the cell(s); and
(c) determining the level of expression and/or secretion of the at least biomarker,
   wherein the level of expression, activity and/or secretion is indicative of the biological activity and/or therapeutic potential of the glucan.

In a third aspect the present invention provides a method for administering to a subject in need thereof at least one cell, the method comprising the steps of:
(a) isolating the at least one cell from the subject, wherein the cell is capable of being stimulated by the glucan;
(b) contacting the at least one cell with an effective amount of the glucan and incubating for a period of time and under suitable conditions sufficient to stimulate the cell(s) thereby inducing a change in the level of expression, activity and/or secretion of at least biomarker from the cell(s); and subsequently
(c) re-introducing the at least one cell into the subject.

In a fourth aspect, the present invention provides a composition comprising at least one cell stimulated in accordance with the method of the first or second aspect.

In a fifth aspect, the present invention provides the use of a glucan for the ex vivo stimulation of at least one cell isolated from a subject, wherein said stimulation is sufficient to induce an alteration in the level of expression, activity and/or secretion of at least one biomarker from the cell(s).

The present invention also provides methods of treatment comprising administering glucans tested in accordance with methods of the invention and pharmaceutical compositions comprising glucans tested in accordance with methods of the invention.

The biomarker may be a biologically active molecule. As such the present invention also provides methods of treatment comprising administering bioactive biomarkers resulting from stimulation of cells in accordance with the above aspects and embodiments, and pharmaceutical compositions comprising such bioactive biomarkers.

### BRIEF DESCRIPTION OF THE DRAWINGS

The present invention will now be described, by way of non-limiting example only, with reference to the accompanying drawings.

FIG. 1. Dose dependent TNF-α release from MPB-M macrophage cultures (CD14+) in the presence of Glucoprime™ (1GP lots), Baker's yeast or endotoxin after 24 hours.

FIG. 2. TNF-α release from human donor-derived macrophages (Donor 70) stimulated ex vivo with 100 mg/mL Glucoprime™ (1GP lots), Baker's yeast or endotoxin wherein lymphocytes were removed from the culturing monocytes after either 24 hours (A) or 14 days (B).

FIG. 3. TNF-α release from human donor-derived macrophages (Donor 71) stimulated ex vivo with 100 mg/mL Glucoprime™ (1GP lots), Baker's yeast or endotoxin wherein lymphocytes were removed from the culturing monocytes after either 24 hours (A) or 14 days (B).

FIG. 4. Time-dependent reduction in TNF-α release from human donor-derived macrophages following Glucoprime™ stimulation.

FIG. 5. Time-dependent TNF-α release from MPB-M macrophage cultures (CD14+) are varying concentrations of Glucoprime™ lot 1GP06.002 at 2 hrs, 4 hrs and 24 hrs.

FIG. 6. IL-8 (A) and GM-CSF (B) production in two human donor-derived macrophages stimulated with 100 μg/mL Glucoprime™. Bars 1 and 4, Glucoprime™ lot 1GP06.002; bars 2 and 5, Glucoprime™ lot 1GP002/98; bars 3 and 6, no Glucoprime™ control. Bars 1 to 3 represent macrophages from a first donor, bars 4 to 6 representing macrophages from a second donor.

### DETAILED DESCRIPTION OF THE INVENTION

The articles “a” and “an” are used herein to refer to one or to more than one (i.e., to at least one) of the grammatical object of the article. By way of example, “an element” means one element or more than one element.

Throughout this specification and the claims which follow, unless the context requires otherwise, the word “comprise”, and variations such as “comprises” or “comprising”, will be understood to imply the inclusion of a stated integer or step or group of integers or steps but not the exclusion of any other integer or step or group of integers or steps.

As used herein the term “biomarker” means any molecule or compound the activity, presence, absence, altered expression, altered cellular distribution or altered secretion of which can be determined, either qualitatively or quantitatively in response to the presence of the glucan. The biomarker may be a direct or indirect product of the cell incubated with the glucan. The biomarker may be any suitable molecule or compound, including a nucleic acid, peptide, protein, lipid, or saccharide.

As used herein the term “effective amount” is used in two contexts, ex vivo and in vivo. In the ex vivo context, an “effective amount” refers to a suitable amount of glucan required to stimulate cells capable of being stimulated by glucans to a sufficient extent to induce an alteration in the level of expression, activity and/or secretion of a biomarker in a cell. The exact amount required will vary from case to case depending on factors such as the nature of the cells, the amount or concentration of cells cultured, the length of time in which the glucan is incubated with the cells, the glucan being used and the form in which the glucan is presented. Thus, it is not possible to specify an exact “effective amount” However, for any given case, an appropriate “effective amount” may be determined by one of ordinary skill in the art using only routine experimentation. In the in vivo context, an “effective amount” includes within its meaning a non-toxic but sufficient amount or dose of an agent or compound to provide the desired therapeutic effect. The exact amount or dose required will vary from subject to subject depending on factors such as the species being treated, the age and general condition of the subject, the severity of the condition being treated, the particular agent being administered and the mode of administration and so forth. Thus, it is not possible to specify an exact “effective amount” However, for any given case, an appropriate “effective amount” may be determined by one of ordinary skill in the art using only routine experimentation.

The term “expression” as used herein refers interchangeably to expression of a gene or gene product, including the encoded protein. Expression of a gene may be determined, for example, by measuring the production of messenger RNA (mRNA) transcript levels. Expression of a polypeptide gene product may be determined, for example, by immunoassay using an antibody(ies) that bind with the polypeptide.

As used herein the term “glucan” includes both a glucan molecule(s) in a purified form or (for example as an isolated molecule) and a glucan present in a composition or formulation. Thus, for the present purposes, the glucan may be associated with one or more additional components, which components are typically not active agents in their own right.

As used herein the term “isolated” means that the cell or cells in question have been removed from their host, and associated material (other cells or extracellular material) reduced or eliminated. Essentially, it means the object cell type is the predominant cell type present. However the “isolated” cell(s) need not be completely free of extraneous material or impurities provided the extraneous material or impurities do not prevent the ex vivo culturing of said cell(s). The terms “isolated” and “purified” may be used interchangeably in the context of the present invention.

The term “response” as used herein in the context of a subject's “response” refers to both clinical response and cellular response. That is, in accordance with the invention a subject's response to the administration of a glucan may be characterised by, or assessed in terms of, the clinical response of the subject, for example as determined by changes in any one or more symptoms of a condition suffered by the subject. Alternatively or in addition, the response of the subject may be assessed or measured at the molecular or cellular level, for example in terms of altered gene expression, or changes in the level of production and/or secretion of molecules such as signalling molecules or extracellular matrix constituents.

As used herein the term “stimulated” means that the cells contacted with the glucan are “stimulated” or “activated” such that the glucan, directly or indirectly, induces a change in the level of expression, activity and/or secretion of a biomarker in the cell when compared to the level of expression, activity and/or secretion observed in the absence of the glucan. The terms “stimulated” and ‘activated” may be used interchangeably in the context of the present invention. The “stimulation” or “activation” of the cells may result in a decrease or increase in the level of expression, activity and/or secretion of the biomarker. Typically, the stimulation or activation implies an increase in said level, although this is not necessarily the case in all embodiments.

As used herein the term “subject” includes humans, primates, livestock animals (eg. sheep, pigs, cattle, horses, donkeys), laboratory test animals (eg. mice, rabbits, rats, guinea pigs), companion animals (eg. dogs, cats) and captive wild animals (eg. foxes, kangaroos, deer). Typically, the mammal is human or a laboratory test animal. Even more typically, the mammal is a human

The term “therapeutic potential” as used herein refers to the potential ability of a glucan to effect a “response” in a subject to which the glucan is administered. Typically the “therapeutic potential” of a glucan is associated with the biological activity of the glucan, which in turn may be determined by a number of means known to those skilled in the art including the ability of the glucan to stimulate the release of cytokines and/or growth factors from macrophages or to indirectly stimulate the production of collagen from fibroblasts. However, more broadly, the term “therapeutic potential” refers to the ability of the glucan to treat a particular condition in a subject. In this context, the term “treat” will be understood to have the meaning provided herein.

As used herein the terms “treating”, “treatment”, “preventing” and “prevention” refer to any and all uses which remedy a condition or symptoms, prevent the establishment of a condition or disease, or otherwise prevent, hinder, retard, or reverse the progression of a condition or disease or other undesirable symptoms in any way whatsoever. Thus the terms “treating” and “preventing” and the like are to be considered in their broadest context. For example, treatment does not necessarily imply that a patient is treated until total recovery.

The present invention provides, inter alia, methods for predicting the response of a subject to the administration of a glucan, and for evaluating the biological activity and/or therapeutic potential of a glucan. The methods described and contemplated herein therefore play an important role in determining the clinical efficacy of glucan compositions for administration to subjects in need thereof, and those skilled in the art will appreciate that by evaluating the therapeutic potential and/or biological activity of a glucan prior to its administration, clinical response can be improved.

Also disclosed herein is the use of a microparticulate beta-(1,3)(1,6) glucan for stimulating a cell population to produce and/or secrete a biomarker, and to uses of cells so stimulated.

A particular application of the present invention is in so-called “personalized medicine”. The term “personalized medicine” is used herein in its broadest context to refer to the tailoring of pharmaceutical compositions and medicines for particular individuals based on and taking into consideration knowledge of the individual's phenotype and/or genotype. Thus, in selecting a composition or medicine to be administered to any particular individual, use is made of information such as the individual's medical history, clinical data and/or the individual's genotype in an attempt to ensure that the composition or medicine is particularly suited to the individual at the time of administration. “Personalized medicine” has the potential to revolutionise the provision of healthcare, however to date little success has been achieved. Embodiments of the present invention provide novel avenues and approaches for the development and delivery of personalized medicine.

The bioassay methodologies described herein may also be advantageously used to determine levels of contamination of batches of glucan products, where such contamination (e.g. endotoxin) may interfere with biological activity, therapeutic efficacy and/or therapeutic suitability of the glucan. The present inventors have developed a novel method for preparing a glucan product substantially free of endotoxin contamination, as described in co-pending U.S. application No. 61/029,739.

In one aspect, there is provided a method for predicting the response of a subject to administration of a glucan prior to said administration, the method comprising:
(a) isolating at least one cell from the subject, wherein the cell is capable of being stimulated by the glucan;
(b) contacting the at least one cell with the glucan and incubating for a period of time and under suitable conditions sufficient to stimulate the cell(s) thereby inducing a change in the level of expression, activity and/or secretion of one or more biomarkers from the cell(s); and
(c) determining the level of expression and/or secretion of the at least biomarker,
   wherein the level of expression, activity and/or secretion is predictive of the response of the subject to administration of the glucan.

The cells employed in accordance with embodiments of the present invention may be isolated or prepared from the subject by any suitable method known in the art. A variety of such techniques and methods are available and well known to those skilled in the art, and the scope of the present invention is not limited by the method adopted. The method of isolation or purification may differ depending on, for example, the cell type to be isolated or purified, the number of cells to be isolated or purified, the tissue or site in the subject from which the cells are derived and the nature of the subject from which the cells are derived. In an embodiment, monocytes are isolated from the subject. These may be derived from any suitable sample, typically a blood sample comprising peripheral blood mononuclear cells (PBMCs). Within a sample of PBMCs, monocytes are typically found in the presence of lymphocytes. The monocytes may be removed from the lymphocytes prior to ex vivo culture of the monocytes. Alternatively, the monocytes may be cultured in the presence of the lymphocytes prior to incubation of the monocytes with the glucan.

The nutritive medium in which the cells are cultured may be any suitable medium capable of sustaining the cells. By way of example only, the medium may be RPMI medium or Dulbecco's modified Eagle medium. The medium may contain various supplements as desired or required for the particular cell type to be cultured and/or as required or beneficial for expression, activity or secretion of the biomarker of interest. For example, in the case of monocytes and macrophages the medium may comprise RPMI medium supplemented with fetal bovine serum (FBS). The medium may also comprise one or more of human sera, amino acids (such as L-glutamine) and antibiotics (such as streptomycin). Where monocytes are isolated from the subject and differentiated ex vivo to generate macrophages, the monocytes may be cultured with differentiation-inducing factors such as granulocyte-macrophage colony stimulating factor (GM-CSF) and interleukin-6 (IL-6).

In general the incubation time of the glucan with the cells is determined by the time required for the cell culture to respond to the glucan, specifically the time required to elicit a change in the level of expression, activity and/or secretion of one or more biomarkers. The incubation time in the presence of the glucan may be from several minutes to several days, for example from abut 10 minutes to 5 days, or from about 30 minutes to 5 days, from about 60 minutes to 4 days, from about 6 hours to 3 days, from about 12 hours to 2 days.

It will be appreciated by those skilled in the art that the cells may be cultured ex vivo for any suitable period of time prior to the addition of the glucan. This culture time may be from several minutes to several days, for example from abut 10 minutes to 5 days, or from about 30 minutes to 5 days, from about 60 minutes to 4 days, from about 6 hours to 3 days, from about 12 hours to 2 days.

The biomarker may be any molecule or compound the activity, presence, absence, altered expression, altered cellular distribution or altered secretion of which can be determined, either qualitatively or quantitatively in response to the presence of the glucan. The nature and identity of the biomarker to be measured will depend on a number of factors including, for example, the nature of the cells to be stimulated by the glucan, the glucan used and the intended use for the cells or glucan. In an embodiment, the cells are monocytes or macrophages and the biomarker is a cytokine, chemokine or growth factor. By way of example only, the biomarker may be a cytokine or growth factor selected from tumor necrosis factor-alpha (TNF-α), interleukin-1 (IL-1), interleukin-1-beta (IL-1β), interleukin-6 (IL-6), interleukin-8 (IL-8), granulocyte macrophage colony stimulating factor (GM-CSF), fibroblast growth factor (FGF), interferon-gamma (IFN-γ), keratinocyte growth factor (KGF), matrix metalloproteinase 1 (MMP-1), matrix metalloproteinase 2 (MMP-2), transforming growth factor-beta 1 (TGF-β1), and vascular endothelial growth factor (VEGF). In a particular embodiment the cytokine is TNF-α or IL-8, more particularly TNF-α.

Biomarker expression may be determined by any suitable means well known to those skilled in the art. Expression may be determined at the transcriptional level, for example determining the presence and/or amount of mRNA transcripts. Alternatively or in addition, expression may be determined or the translational level, for example involving detection of the presence and/or amount of polypeptides, precursors or derivatives thereof. Detection of polypeptides, precursors or derivatives thereof may be via a number of means including immunological means such as enzyme-linked immunosorbent assays (ELISA), mass spectrography, or chromatography. Similarly, such methods for the detection of biomarker polypeptides, precursors or derivatives thereof may be used to determine the level of secretion from cells to the extracellular environment. Accordingly, the determination of biomarker “expression and/or secretion” includes within its scope the detection or quantification of gene expression, protein expression, post-transcriptional and post-translational modifications and/or or measurement of secretion. The analysis of expression and/or secretion may be carried out directly in the culture medium and/or in the cells to determine the amount of non-secreted biomarker.

The biomarker may be a biologically active molecule. As such it is contemplated that in certain embodiments of the invention the biomarker to be detected may be isolated or purified from the cells or culture medium for subsequent therapeutic or other application. For example it may be desirable to utilise cells isolated from an individual to produce a biologically (e.g. therapeutically) active biomolecule for re-administration to the individual, wherein the cells are stimulated to produce the biomolecule ex vivo in the presence of a glucan.

Typically, for the purpose of evaluating the biological activity and/or therapeutic potential of a glucan, the analysis of biomarker expression, activity and/or secretion following incubation of cells in the presence of glucan is compared to the level of expression, activity and/or secretion following incubation of the equivalent cells in the absence of the glucan. Thus methods of the invention may comprise a step of assessing the results of the analysis of expression, activity and/or secretion in relation to a control.

Typically the glucan is a microparticulate glucan, more typically a microparticulate branched beta-(1,3)(1,6) glucan such as poly-(1,3)-beta-D-glucopyranosyl-(1,6)-beta-D-glucopyranose. The glucan may be a microparticulate glucan prepared in accordance with the process as described in U.S. Pat. No. 6,242,594 (Kelly; the disclosure of which is incorporated herein by reference in its entirety). However those skilled in the art will appreciate that the scope of the present invention is not limited thereto.

Embodiments of the present invention also find application in glucan manufacturing processes as part of batch control and quality assurance procedures. That is, the bioassay methods of the invention may be used to evaluate the level of biological activity and thus the therapeutic potential of any particular batch of glucan product, wherein the ability of the glucan product to stimulate or activate cells, such as monocytes and macrophages, as determined by the level of expression, activity and/or secretion of a biomarker such as TNF-α, is indicative of the biological activity (and hence the integrity) of the glucan product. Those skilled in the art will readily appreciate that such evaluation is of particular importance in screening and assessing the viability and efficacy of a product or composition destined for therapeutic administration. The viability and/or efficacy of a product or composition may decrease over time and may be affected by a number of factors including storage conditions (such as temperature, humidity), variability of components (both consumables and equipment) used in the glucan manufacturing process or composition preparation process, and modifications in the glucan manufacturing process or composition preparation process.

It will therefore also be appreciated by those skilled in the art that embodiments of the present invention may be utilised in assessing the effectiveness and suitability of glucan manufacturing processes and processes for the production of pharmaceutical compositions and formulations comprising the glucan. For example, it may be desirable to test a novel manufacturing process, to modify an existing manufacturing process, scale-up production or automate production. In these circumstances the present invention provides means for reliably determining the validity of the alterations and their impact on the activity of the glucan produced. Similarly, the invention provides means for the reliable determination of the effect of formulating a composition comprising a glucan, for example, the effect of various diluents, adjuvants, carriers or other ingredients on activity of the glucan, or the effect on glucan activity of formulating the composition for a particular mode or route of delivery.

Embodiments of the invention also find application in the evaluation of the biological activity of and the potential for therapeutic application of novel glucan molecules and formulations.

In accordance with the aspects and embodiments of the present invention the subject may be suffering from or be predisposed to any condition the treatment of which may be effected by the administration of a glucan, typically a microparticulate beta-(1,3)(1,6) glucan. The subject may, for example, suffer from a skin wound or lesion, or a connective tissue disease or injury. The subject may require, for example, treatment of: a surgical wound, burn wound, chronic ulcer, pressure sore, bed sore, diabetic ulcer, venous ulcer, burn or other wound requiring closure; ultraviolet light-induced skin damage; infection; a condition requiring tissue augmentation such as urinary incontinence; skin wrinkles or blemishes; tissue defects following trauma or surgery; connective tissue damage or injury including injuries to tendons and ligaments; bone fractures, joint damage including to artificial joints; or otherwise require the enhancement of fixation of implanted orthopedic devices, including pins, screws and artificial joints.

Also contemplated by the present invention are methods of treatment wherein glucan, cells stimulated ex vivo with glucan, or the biomarker detected in accordance with aspects and embodiments of the invention are administered to subjects in need thereof. Typically, where cells are administered such administrations are autologous, that is the cells are re-introduced into the subject from which the cells were isolated.

Agents (glucan, cells or biomarker) may be administered in accordance with the present invention in the form of pharmaceutical compositions, which compositions may comprise one or more pharmaceutically acceptable carriers, excipients or diluents. Such compositions may be administered in any convenient or suitable route such as by parenteral, oral, nasal or topical routes. In circumstances where it is required that appropriate concentrations of the desired agent are delivered directly to the site in the body to be treated, administration may be regional rather than systemic. Regional administration provides the capability of delivering very high local concentrations of the desired agent to the required site and thus is suitable for achieving the desired therapeutic or preventative effect whilst avoiding exposure of other organs of the body to the compound and thereby potentially reducing side effects.

It will be understood that the specific dose level of a composition of the invention for any particular individual will depend upon a variety of factors including, for example, the activity of the specific agents employed, the age, body weight, general health and diet of the individual to be treated, the time of administration, rate of excretion, and combination with any other treatment or therapy. Single or multiple administrations can be carried out with dose levels and pattern being selected by the treating physician. A broad range of doses may be applicable. Considering a patient, for example, from about 0.1 mg to about 1 mg of agent may be administered per kilogram of body weight per day. Dosage regimens may be adjusted to provide the optimum therapeutic response. For example, several divided doses may be administered daily, weekly, monthly or other suitable time intervals or the dose may be proportionally reduced as indicated by the exigencies of the situation.

Generally, an effective dosage is expected to be in the range of about 0.0001 mg to about 1000 mg per kg body weight per 24 hours; typically, about 0.001 mg to about 750 mg per kg body weight per 24 hours; about 0.01 mg to about 500 mg per kg body weight per 24 hours; about 0.1 mg to about 500 mg per kg body weight per 24 hours; about 0.1 mg to about 250 mg per kg body weight per 24 hours; about 1.0 mg to about 250 mg per kg body weight per 24 hours. More typically, an effective dose range is expected to be in the range about 1.0 mg to about 200 mg per kg body weight per 24 hours; about 1.0 mg to about 100 mg per kg body weight per 24 hours; about 1.0 mg to about 50 mg per kg body weight per 24 hours; about 1.0 mg to about 25 mg per kg body weight per 24 hours; about 5.0 mg to about 50 mg per kg body weight per 24 hours; about 5.0 mg to about 20 mg per kg body weight per 24 hours; about 5.0 mg to about 15 mg per kg body weight per 24 hours.

The agent may be administered in the form of pharmaceutically acceptable nontoxic salts, such as acid addition salts or metal complexes, e.g. with zinc, iron or the like (which are considered as salts for purposes of this application). Illustrative of such acid addition salts are hydrochloride, hydrobromide, sulphate, phosphate, maleate, acetate, citrate, benzoate, succinate, malate, ascorbate, tartrate and the like. Examples of pharmaceutically acceptable carriers or diluents are demineralised or distilled water; saline solution; vegetable based oils such as peanut oil, safflower oil, olive oil, cottonseed oil, maize oil, sesame oil, arachis oil or coconut oil; silicone oils, including polysiloxanes, such as methyl polysiloxane, phenyl polysiloxane and methylphenyl polysolpoxane; volatile silicones; mineral oils such as liquid paraffin, soft paraffin or squalane; cellulose derivatives such as methyl cellulose, ethyl cellulose, carboxymethylcellulose, sodium carboxymethylcellulose or hydroxypropylmethylcellulose; lower alkanols, for example ethanol or iso-propanol; lower aralkanols; lower polyalkylene glycols or lower alkylene glycols, for example polyethylene glycol, polypropylene glycol, ethylene glycol, propylene glycol, 1,3-butylene glycol or glycerin; fatty acid esters such as isopropyl palmitate, isopropyl myristate or ethyl oleate; polyvinylpyrridone; agar; carrageenan; gum tragacanth or gum acacia, and petroleum jelly. Typically, the carrier or carriers will form from 10% to 99.9% by weight of the compositions.

Topical formulations typically comprise an active ingredient together with one or more acceptable carriers, and optionally any other therapeutic ingredients. Formulations suitable for topical administration include liquid or semi-liquid preparations suitable for penetration through the skin to the site of where treatment is required, such as liniments, lotions, creams, ointments or pastes, and drops suitable for administration to the eye, ear or nose.

Lotions according to the present invention include those suitable for application to the skin or eye. An eye lotion may comprise a sterile aqueous solution optionally containing a bactericide and may be prepared by methods similar to those described above in relation to the preparation of drops. Lotions or liniments for application to the skin may also include an agent to hasten drying and to cool the skin, such as an alcohol or acetone, and/or a moisturiser such as glycerol, or oil such as castor oil or arachis oil.

Creams, ointments or pastes according to the present invention are semi-solid formulations of the active ingredient for external application. They may be made by mixing the active ingredient in finely-divided or powdered form, alone or in solution or suspension in an aqueous or non-aqueous fluid, with a greasy or non-greasy basis. The basis may comprise hydrocarbons such as hard, soft or liquid paraffin, glycerol, beeswax, a metallic soap; a mucilage; an oil of natural origin such as almond, corn, arachis, castor or olive oil; wool fat or its derivatives, or a fatty acid such as stearic or oleic acid together with an alcohol such as propylene glycol or macrogols.

Pharmaceutical forms suitable for injectable use include sterile aqueous solutions (where water soluble) or dispersions and sterile powders for the extemporaneous preparation of sterile injectable solutions. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of superfactants. The preventions of the action of microorganisms can be brought about by various antibacterial and antifungal agents, for example, parabens, chlorobutanol, phenol, sorbic acid, thimerosal and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilisation. Generally, dispersions are prepared by incorporating the various sterilised active ingredient into a sterile vehicle which contains the basic dispersion medium and the required other ingredients from those enumerated above. In the case of sterile powders for the preparation of sterile injectable solutions, the preferred methods of preparation are vacuum drying and the freeze-drying technique which yield a powder of the active ingredient plus any additional desired ingredient from previously sterile-filtered solution thereof.

When the active ingredients are suitably protected they may be orally administered, for example, with an inert diluent or with an assimilable edible carrier, or it may be enclosed in hard or soft shell gelatin capsule, or it may be compressed into tablets, or it may be incorporated directly with the food of the diet. For oral therapeutic administration, the active compound may be incorporated with excipients and used in the form of ingestible tablets, buccal tablets, troches, capsules, elixirs, suspensions, syrups, wafers, and the like. Such compositions and preparations should contain at least 1% by weight of active compound. The percentage of the compositions and preparations may, of course, be varied and may conveniently be between about 5 to about 80% of the weight of the unit. The amount of active compound in such therapeutically useful compositions in such that a suitable dosage will be obtained. Preferred compositions or preparations according to the present invention are prepared so that an oral dosage unit form contains between about 0.1 μg and 2000 mg of active compound.

The tablets, troches, pills, capsules and the like may also contain the components as listed hereafter: a binder such as gum, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin may be added or a flavouring agent such as peppermint, oil of wintergreen, or cherry flavouring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavouring such as cherry or orange flavour. Of course, any material used in preparing any dosage unit form should be pharmaceutically pure and substantially non-toxic in the amounts employed. In addition, the active compound(s) may be incorporated into sustained-release preparations and formulations.

The present invention contemplates combination therapies, wherein agents as described herein are coadministered with other suitable agents which may facilitate the desired therapeutic or prophylactic outcome. For example, in the context of asthma, one may seek to maintain ongoing anti-inflammatory therapies in order to control the incidence of inflammation whilst employing agents in accordance with embodiments of the present invention. By “coadministered” is meant simultaneous administration in the same formulation or in two different formulations via the same or different routes or sequential administration by the same or different routes. By “sequential” administration is meant a time difference of from seconds, minutes, hours or days between the administration of the two types of molecules. These molecules may be administered in any order.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

The present invention will now be described with reference to the following specific examples, which should not be construed as in any way limiting the scope of the invention.

### EXAMPLES

### Example 1

### TNF-α Release from Macrophages Stimulated with Glucoprime™

Briefly, monocytes were isolated from human blood and stimulated with Granulocyte Macrophage Colony Stimulation Factor (GM-CSF) and Interleukin-6 (IL-6) to more completely differentiate the monocytes into macrophages. Cells were culture in a basal medium supllmented with 10% fetal bovine serum (FBS) as described below. Non-adherent cells from the peripheral blood mononuclear cell cultures (i.e. lymphocytes) were removed after 24 hours. Cultures were then stimulated with 100 μg/mL, 100 μg/mL or 100 μg/mL Glucoprime™ (microparticulate beta-(1,3)(1,6) glucan derived from Saccharomyces cerevisiae), 100 μg/mL, 100 μg/mL or 100 μg/mL Baker's Yeast or with 100 ng/mL of Endotoxin (positive control). A tumor necrosis factor alpha (TNF-α) enzyme-linked immunosorbant assay (ELISA) was performed on the culture supernatants following a 24-hour incubation with the glucans or controls. Additionally, Granulocyte Colony Stimulating Factor Mobilized Peripheral Blood Cluster Differentiation (CD)14+ Monocytes (MPB-M) were thawed, cultured, and stimulated with Glucoprime™. Here, monocyte-to-macrophage differentiation over seven days was stimulated by again adding GM-CSF and IL-6. After 7-days in culture the cells were stimulated with Glucoprime™ or controls. A TNF-α ELISA was performed on the culture supernatants to measure bioactivity. An immunofluorescence analysis was performed on a subset of these cultures for the macrophage cell surface marker CD14.

The methodology is described in more detail below.

Culture Medium

‘MØ Medium-10% FBS’ was formulated by adding 50 mL of Fetal Bovine Serum (FBS) and 5.0 mL of 200 mM L-Glutamine (Lonza 17-605E) to 450 mL of RPMI 1640. In cultures established with monocytes isolated from freshly obtained human donor blood and in MPB-M cultures, 10 μg/mL stocks of GM-CSF and IL-6 were added to final concentrations of 10 ng/mL each in the ‘MØ Medium-10% FBS’ before addition to the cell cultures.

Whole Blood Collection and White Blood Cell Isolation

Human blood was collected from two normal paid volunteers under informed consent via venipuncture into BD Vacutainer® CPT™ Cell Preparation Tubes containing Sodium Citrate (BD 362760). The identity of the donors was kept confidential by coding the blood samples. The tubes were inverted to mix the blood with the anti-coagulant. The tubes were centrifuged at 1800 RCF for 20 minutes at room temperature. Following centrifugation the tubes were inverted 5-10 times. Granulocytes and red blood cells had been pulled through the vacutainer's gel plug and the mononuclear cells, plasma, and platelets remained above the plug as a clear yellow suspension. The suspension was mixed by inversion and centrifuged at 300 RCF for 10 minutes at room temperature (twice). The resulting cells were resuspended in culture medium.

White Blood Cell Culture

Peripheral blood mononuclear cells (PBMCs) from the freshly donated blood were seeded in 1 mL volumes at a concentration of 2.0×10⁶ cells per well in 24 well tissue culture plates. After 24 hours culturing at 37° C. with 5% CO₂ during which time the monocytes adhere to the culture surface, the non-adherent lymphocytes were removed from half of the cultures in each plate. 0.9 mL of medium was replaced into the cultures and supplemented with 0.1 mL of 100 ng/mL of GM-CSF and IL-6 for a final concentration of 10 ng/mL. The culture plates were incubated at 37° C. with 5% CO₂ for 14 days to allow for monocyte differentiation into macrophages. The lymphocytes in the other half of the cultures were not removed. They were incubated as monocyte/lymphocyte co-cultures for the entire 14-day differentiation incubation.

Media was replenished twice per week by removing 0.80 mL of the spent culture medium and replacing it with 0.70 mL fresh culture medium plus 0.1 mL of 100 ng/mL of GM-CSF and IL-6 for a final in culture concentration of 10 ng/mL of each cytokine. On day 14 all the non-adherent cells from the cultures were removed. The adherent monocytes were washed with 1 mL of PBS and 0.9 mL of the MØ medium was replaced into the cultures and stimulated with the appropriate glucan.

Peripheral Blood Cluster Differentiation (CD14+) Monocyte (MPB-M) Cultures

A frozen ampoule of MPB-M cells (LONZA) was placed in a 37° C. water bath and gently swirled until just thawed. The contents of the ampoule were mixed with 9 mL of MØ Medium-10% FBS and centrifuged at 210 RCF for 5 minutes at room temperature. Following centrifugation, the supernatant was aspirated and the cell pellet was resuspended in 5 mL of MØ Medium-10% FBS. A cell count was performed using Trypan Blue. The cells were seeded in 24 well plates at 5.0×10⁵ cells per well. GM-CSF and IL-6 were added to each well to make a final concentration of 10 ng/mL of each cytokine. The culture plates were incubated at 37° C. with 5% CO₂ for 7 days. The cultures were fed on day 3 or 4 as described above. On day 7, the non-adherent cells from the cultures were removed as stated above for the white blood cell cultures and the cultures were stimulated with the appropriate glucan.

Glucans

Glucoprime™ suspensions were prepared as stock solutions in PBS to reach a 1 mg/mL concentration. Glucoprime™ suspensions and corresponding placebos were also prepared from gel formulations. The gels (Glucoprime™ and placebo) were prepared in MØ Medium-10% FBS to reach a 1 mg/mL concentration of gel per mL of MØ Medium-10% FBS, which is equivalent to a 0.01 mg/mL Glucoprime™ concentration per mL of MØ Medium-10% FBS. The spent medium in the culture well was removed and replaced with 1 mL of the 1 mg/mL gel suspension in MØ Medium-10% FBS for a final concentration of 0.01 mg/mL per well. Glucan consisting of Baker's Yeast (Sigma G5011) suspensions were also prepared as stock solutions in PBS to reach a 1 mg/mL concentration.

Glucan Addition to Macrophage Cultures

For macrophages derived from differentiated monocytes isolated from human volunteers, after removal of the non-adherent cells in the 24 well plate cultures, 0.90 mL of MØ Medium-10% FBS was added. Glucoprime™ suspension was added to the corresponding wells for a final concentration of 100 μg/mL. Positive controls (for stimulation) consisting of 100 μg/mL of Baker's Yeast (Sigma G5011) and 100 ng/mL of Endotoxin (Lonza N185) were also included on each plate. For the negative control (‘Control No Stim.’), 0.1 mL PBS was added. The cultures were observed microscopically. Photographs were taken of the cells at 24 hours post stimulation and immunofluorescence staining was conducted to measure the cell surface marker CD14. Supernatants were harvested from each well at 4 hours or 24 hours post stimulation and immediately frozen at −30° C. for later analysis by ELISA.

In the case of MPB-M cultures, after removal of the non-adherent cells in the 24-well plates, 0.90 mL of MØ Medium-10% FBS was added to all the wells. 0.1 mL of 1 mg/mL Glucoprime™ suspension was added to the corresponding wells for a final concentration of 100 μg/mL. Positive controls (for stimulation) consisting of 100 μg/mL of Baker's Yeast (Sigma G5011) and 100 ng/mL of Endotoxin (Lonza) were also included on each plate. For the negative control, 0.1 mL PBS was added. The cultures were observed microscopically. Supernatants were harvested from each well at 24 hours post stimulation and immediately frozen at −30° C. for later analysis by ELISA.

TNF-α ELISA

TNF-α levels were measured using ELISA kits obtained from R&D Systems, Inc. Macrophage culture supernatants were added to the ELISA plate. All ELISAs were performed according to the manufacturer's instructions. Samples were assayed undiluted or diluted between 1:10 and 1:10,000.

Results

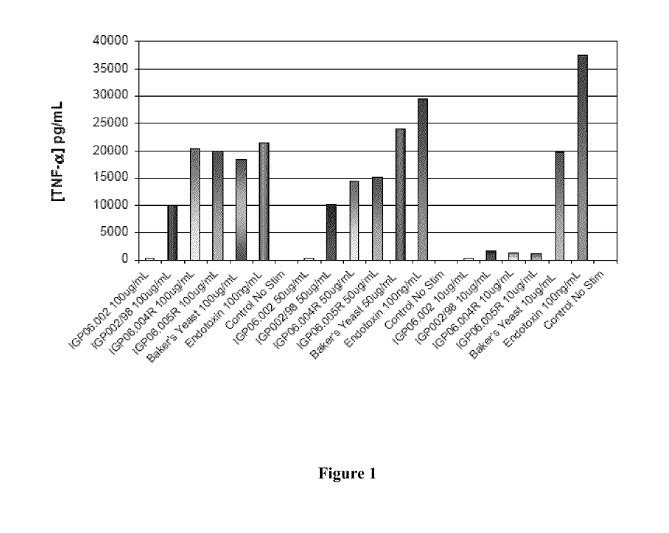

FIG. 1 shows TNF-α release from MPB-M cultures stimulated with Glucoprime™, Baker's yeast or endotoxin. TNF-α was produced in a dose-dependent response to Glucoprime™ stimulation in all lots tested (1GP06.002, 1GP002/98, 1GP06.004R and 1GP06.005R). Maximal TNF-α release was observed in the presence of 100 μg/mL Glucoprime™.

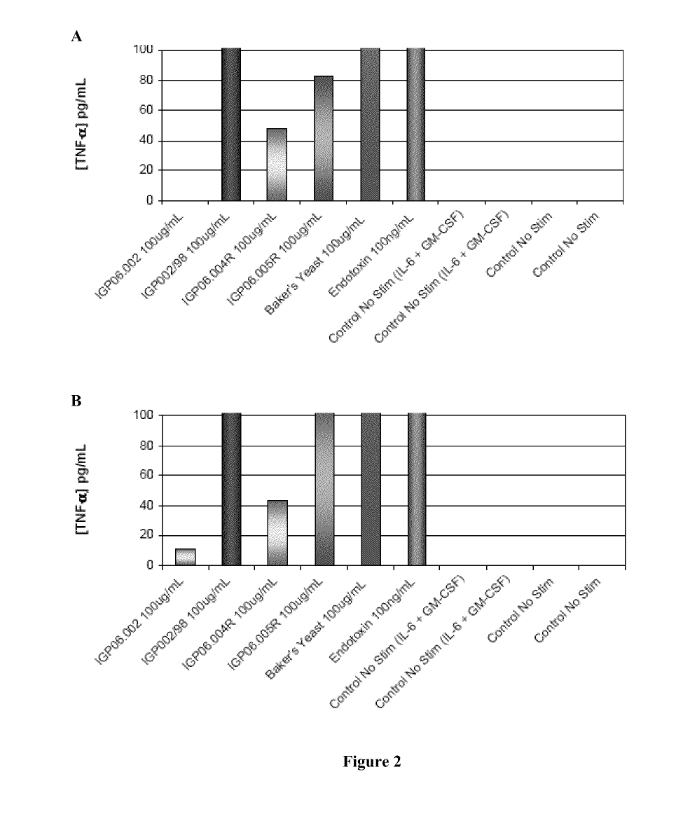

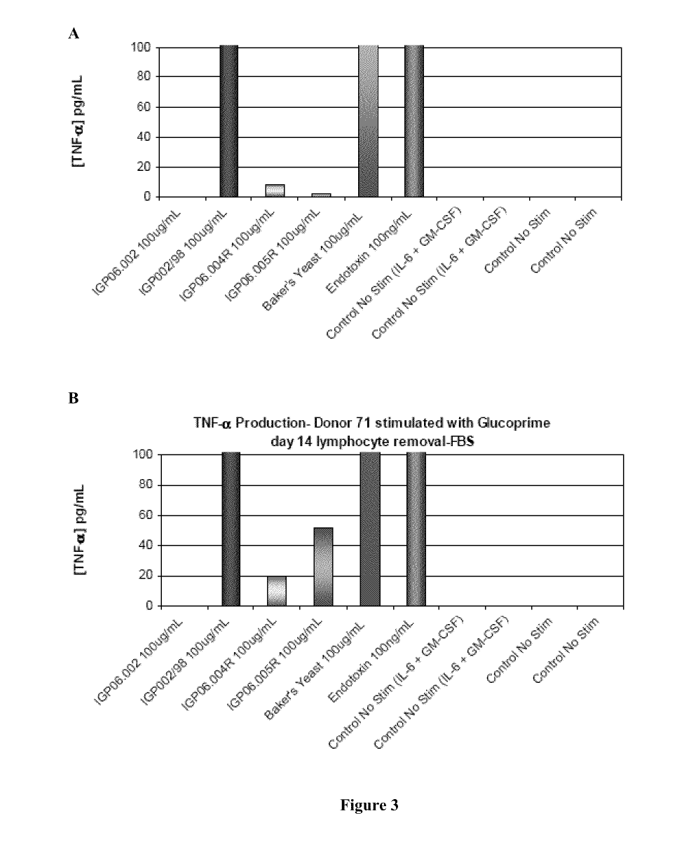

FIGS. 2 and 3 show TNF-α release from macrophages derived from monocytes isolated from blood samples of two human donors, donor 70 (FIG. 2) and donor 71 (FIG. 3). As illustrated, Glucoprime™ successfully stimulated the macrophages of both subjects to release TNF-α. The amount of TNF-α release varied between subjects and between Glucoprime™ lots thereby suggesting the viability of ex vivo bioassays as provided by the present invention for the prediction of a subject's response to Glucoprime™ administration.

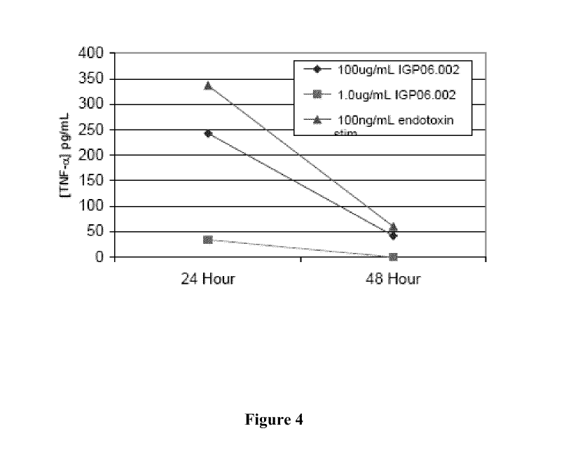

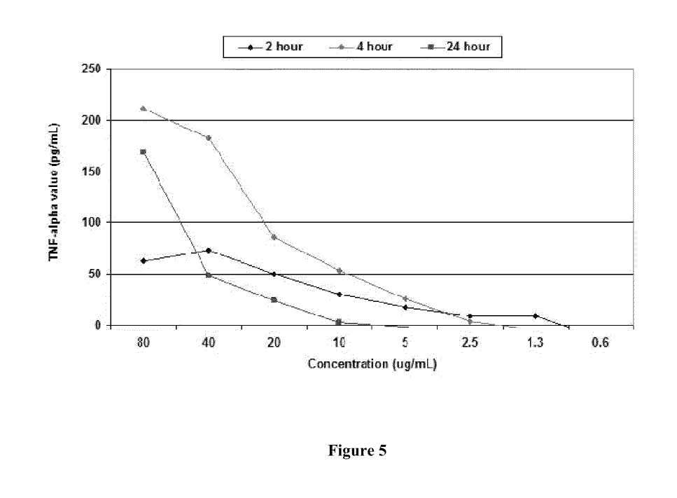

As shown in FIG. 4, a time course assay of TNF-α release from macrophages derived from a human donor illustrates the time-dependent reduction in secreted TNF-α levels following Glucoprime™ addition. A further time-course of TNF-α release from MPB-M cells was conducted in which TNF-α release was determined at varying concentrations of Glucoprime™ at time intervals of 2 hours, 4 hours and 4 hours. For Glucoprime lot 1GP06.002, an illustrative time-course is shown in FIG. 5. It can be seen that there is a dose-dependent stimulation of TNF-α levels at 4 hours and lower concentrations of TNF-α after 24 hours. These data illustrate that methods of the present invention enable not only the detection of secreted TNF-α, but also the time-course of such secretion thereby providing valuable information regarding the biological activity and efficacy of a glucan and providing insight regarding the most appropriate administration of a glucan to achieve a desired therapeutic outcome.

### Example 2

### IL-8 and GM-CSF Release from Macrophages Stimulated with Glucoprime™

Experiments were conducted as per Example 1 above using macrophages derived from monocytes isolated from 2 human donors. Cells were stimulated with 100 μg/mL Glucoprime™ (lots 1GP06.002 and 1GP002/98) and levels of IL-8 and GM-CSF in the supernatant were determined using commercially available ELISAs.

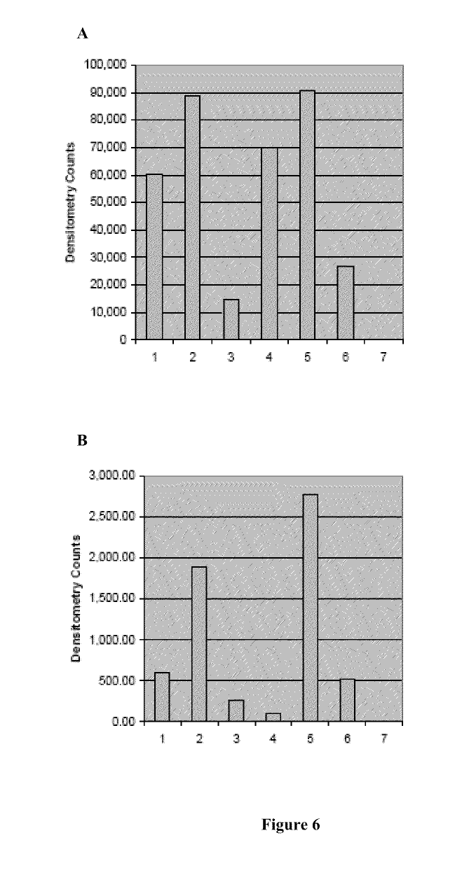

As shown in FIG. 6A, both Glucoprime™ lots stimulated IL-8 production in macrophages from each donor. The absolute level of stimulation (as determined by IL-8 production) differed between Glucoprime™ lots.

In the case of GM-CSF, the macrophages of each donor responded differently to Glucoprime™ addition (FIG. 6B).

### Example 3

### In Vivo Efficacy of Glucoprime™ Demonstrated to Stimulate TNF-α Release

To demonstrate the ability of methods of the present invention to predict efficacy and subject response to glucan administration, Glucoprime™ samples were tested in vitro for their ability to stimulate TNF-α release prior to being administered to subjects in vivo.

TNF-α release was determined as described above in Example 1 for cells stimulated with one of five test samples of Glucoprime™ at varying concentrations between 0.6 μg/ml and 80 μg/ml. The results are shown in Table 1 below comparing the ability of these test samples to stimulate TNF-α release with that of a glucan reference sample (RS), endotoxin and a no glucan (vehicle) control sample.

Test samples of Glucoprime™ exhibiting TNF-α release activity above that of the no glucan vehicle control were subsequently employed in a study of the ability of Glucoprime™ to treat skin wounds in minipigs and were shown to be efficacious (the results of this in vivo study are described in applicant's co-pending U.S. application No. 61/105,525). These results clearly demonstrate that methods in accordance with the present invention are able to successfully determine the clinical effectiveness of glucan samples.

1. A method for predicting the response of a subject to administration of a beta-(1,3)(1,6) glucan prior to said administration, the method comprising:
(a) isolating at least one cell from the subject, wherein the cell is capable of being stimulated by the glucan;
(b) contacting the at least one cell with the glucan and incubating for a period of time and under suitable conditions sufficient to stimulate the cell(s) thereby inducing a change in the level of expression, activity and/or secretion of one or more biomarkers from the cell(s); and
(c) determining the level of expression and/or secretion of the one or more biomarkers,
wherein the level of expression, activity and/or secretion of the one or more biomarkers is predictive of the response of the subject to administration of the glucan.

2. The method of claim 1 wherein the at least one cell capable of being stimulated by the glucan is a macrophage or precursor thereof.

3. The method of claim 2 wherein the precursor is a monocyte.

4. The method of claim 1 wherein the at least one cell isolated from the subject comprises monocytes that are subsequently differentiated in vitro prior to or during incubation with the glucan.

5. The method of claim 1 wherein the subject is suffering from any condition the treatment of which can be effected by administration of the glucan.

6. The method of claim 5 wherein the condition is a skin wound or lesion, or a connective tissue disease or injury.

7. The method of claim 1 wherein the stimulation of the cell(s) by the glucan induces an increase or decrease in the level of expression, activity and/or secretion of the one or more biomarkers relative to the level detected in the absence of the glucan (unstimulated cell(s)).

8. The method of claim 7 wherein the method comprises the further step of comparing the level of expression, activity and/or secretion of the one or more biomarkers in the presence of the glucan with the corresponding level of the same biomarker(s) in the absence of glucan, whereby the difference in the levels of expression, activity and/or secretion is predictive of the subject's response to administration of the glucan.

9. The method of claim 8 wherein the level of expression, activity and/or secretion in the absence of the glucan may be a predetermined control level.

10. The method of claim 1 wherein the predicted response of the subject to administration of the glucan correlates with the biological activity of the glucan as determined by the level of expression, activity and/or secretion of the one or more biomarkers.

11. The method of claim 10 wherein the degree of change in the level of expression, activity and/or secretion of the biomarker(s) in the presence of the glucan is indicative of the biological activity and/or therapeutical potential of the glucan and predictive of the response of a subject to administration of the glucan.

12. The method of claim 1 wherein the stimulation of the cells results in an increase in the level of expression, activity and/or secretion of the one or more biomarkers relative to the level of expression and/or secretion in the absence of the glucan.

13. The method of claim 1 wherein the biomarker is a cytokine, chemokine or growth factor.

14. The method of claim 13 wherein the biomarker is TNF-α.

15. The method of claim 1 wherein the cells are macrophages and the determining step (c) comprises determining the level of secretion of TNF-α from the macrophages.

16. The method of claim 1 wherein the cells are incubated in a suitable nutritive culture medium capable of sustaining the cells, the medium optionally including additional co-factors required or beneficial for expression, activity and/or secretion of the biomarker(s).

17. The method of claim 1 wherein the glucan is derived from yeast cell walls.

18. The method of claim 1 wherein the glucan comprises a particulate or microparticulate beta-(1,3)(1,6) glucan.

19. The method of claim 18 wherein the glucan is microparticulate poly-(1,3)-beta-D-glucopyranosyl-(1,6)-beta-D-glucopyranose.

20. The method of claim 1 wherein the cell(s) are re-introduced into the subject from which they were isolated following the incubation step (b).

21. A method for evaluating the biological activity and/or therapeutic potential of a batch of beta-(1,3)(1,6) glucan product, the method comprising:
(a) isolating at least one cell from a subject, wherein the cell is capable of being stimulated by the glucan;
(b) contacting the at least one cell with a selected batch of glucan product and incubating for a period of time and under suitable conditions sufficient to stimulate the cell(s) thereby inducing a change in the level of expression, activity and/or secretion of one or more biomarkers from the cell(s); and
(c) determining the level of expression and/or secretion of the one or more biomarkers,
wherein the level of expression, activity and/or secretion of the one or more biomarkers is indicative of the biological activity and/or therapeutic potential of the batch of glucan product.

22. A method for administering to a subject in need thereof at least one cell, the method comprising the steps of:
(a) isolating the at least one cell from the subject, wherein the cell is capable of being stimulated by a beta-(1,3)(1,6) glucan;
(b) contacting the at least one cell with an effective amount of a beta-(1,3)(1,6) glucan and incubating for a period of time and under suitable conditions sufficient to stimulate the cell(s) thereby inducing a change in the level of expression, activity and/or secretion of one or more biomarkers from the cell(s); and subsequently
(c) re-introducing the at least one cell into the subject.

23. A composition comprising at least one cell stimulated in accordance with the method of claim 1.

24. (canceled)

25. (canceled)

26. A method of treatment comprising administering to a subject in need thereof bioactive biomarkers resulting from the stimulation of at least one cell wherein the at least one cell is isolated from the subject and stimulated ex vivo with a beta-(1,3)(1,6) glucan, wherein said stimulation is sufficient to induce an alteration in the level of expression, activity and/or secretion of at least one biomarker from the at least one cell.

27. A composition comprising at least one cell stimulated in accordance with the method of claim 2.
